(19) United States
(12) Patent Application Publication
(10) Pub. No.: US 2005/0014135 A1
(43) Pub. Date: Jan. 20, 2005

(54) Method for the selection and identification of peptide or protein molecules by means of phage display

(76) Inventors: Oliver Hill, Neckarsteinach (DE); Holger Ottleben, Heidelberg (DE); Stefan Dickopf, Heidelberg (DE); Klaus Burkert, Heidelberg (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/855,668

(22) Filed: May 27, 2004

Related U.S. Application Data
(63) Continuation of application No. PCT/EP02/13010, filed on Nov. 20, 2002.

(30) Foreign Application Priority Data
Nov. 28, 2001 (DE) DE 10158242.0
May 8, 2002 (DE) DE 10220602.3

(51) Int. Cl. C12Q 1/70

(52) U.S. Cl. 435/5

(57) ABSTRACT

The invention relates to a method for the selection and identification of at least on representative (interaction partner) from a plurality of peptide or protein molecules, which can specifically interact with at least one representative from a plurality of target molecules, forming a bond. The inventive method comprises the following steps: (a) a virus system consisting of a plurality of viruses, wherein each virus respectively presents at least on representative from the plurality of peptide or protein molecules on the surface thereof, is brought into contact with the plurality of target molecules (ligands) which are immobilized on the surface of a solid phase carrier such that they are position addressable in a two-dimensional grade; (b) unbound viruses removed from the surface thereof; and (c) the interaction partner is identified by detection and determination of the position of the bond between the immobilized ligand and the interaction partner presented by the virus with the aid of a marker-free detection method. The described method makes it possible to concentrate viruses presenting interaction partners by means of an optionally cyclic repetition of selection. Optionally, selected interaction partners are recombinantly expressed after identification of the coding nucleotide sequence.

The present invention relates to a method for the selection and identification of peptide or protein molecules by means of phage display.

Selection and identification methods have led to the understanding of numerous biological processes. The conventional experimental approaches used therein to date were cell-based screening and affinity chromatography. Although both techniques are helpful for discovering peptide or protein molecules, a method which couples protein identification with gene isolation is very desirable.

Phage display screening systems follow this approach. The combination of in vitro gene expression techniques with traditional biochemical approaches such as, for example, affinity chromatography, which is used therein offers the possibility of functional gene selection by creating a direct link between natural product affinity and gene structure.

In phage display, genes encoding non-viral proteins or peptides are incorporated into the viral genome such that fusion proteins are generated between the desired non-viral protein or peptide and a viral coat protein. The fusion protein is thereby presented on the surface of the virus during replication of the virus in the host. In a typical phage display library, a plurality of DNA fragments encoding non-viral proteins or peptides are inserted in the viral genome. Viral particles that present a plurality of proteins or peptides on the surface are thus generated.

This phage display library is then brought into contact with a sample immobilised on a carrier. During the following washing step, viruses presenting fusion proteins that interact with the immobilised sample to form a bond are retained on the carrier whereas viruses which do not present interacting fusion proteins are washed away. The interacting viruses are eluted and amplified by infection of a host culture. Repeated amplification and selection rounds may be required in order to obtain a comparatively homogeneous virus population which binds to the immobilised sample with high affinity. The inserted DNA sections of individual virus clones are subsequently sequenced and the amino acid sequences of the interacting proteins or peptides are derived therefrom.

The immobilisation of an interaction partner (ligand) is necessary to enable separation of the virus-ligand complexes from the non-interacting viruses. It furthermore facilitates conduction of the process such as, for example, the performance of washing steps, and may, in connection with a suitable detection method, give information about the presence and strength of the interactions between the interaction partners.

Spherical beads (cross-linked polymers in particle form) are often used as carriers for the virus selection. Beads can, for example, be stacked into affinity columns. A big disadvantage of using these bead affinity columns is that no spatial assignment of the beads is possible. This presents a considerable problem for the automation and miniaturisation of bead-based phage display methods. The complex handling of the beads, for example when carrying out washing steps, is also disadvantageous. A further disadvantage is that clearances form between the spherical beads, in which dead volumes of virus suspension collect.

It would therefore be more advantageous to use a carrier for virus selection which ensures the immobilisation of a plurality of ligands as well as a universal and easy handling, and thus enables an automation and miniaturisation of the method. The geometry of the carrier used for the virus selection plays an important role for the automation and miniaturisation of the method. It is herefor advantageous if the interaction partners immobilised on the carrier are arranged in a regular grid and in a position-addressable manner. It would furthermore be very advantageous for the automation and miniaturisation of the method if both selection and the detection of bonding occurrences could be carried out on the identical surface. In order to enable the easy use of efficient robots for pipetting or the like, the containers should furthermore be open at the top. A microtiter plate or a planar carrier (e.g. a membrane), for example, meet these requirements.

Described in WO 01/02554 is a parallelised method for identifying interaction partners by means of phage display, in which magnetic particles are used to immobilise the ligands. All selections are simultaneously carried out in spatially separated volumes in microtiter plates. The necessary transfer of the magnetic particles occurs in an automated manner by means of a specific magnetic array. The magnetic particles with the ligand-virus complexes are hereby bonded to the magnets of the array and are transferred between the containers. Discrimination between interacting and non-interacting viruses is carried out using an ELISA (Enzyme Linked Immunosorbent Assay) based method in a process following selection.

A big disadvantage in using microtiter plates as the incubation vessel is that the cavities only allow a relatively small sample volume for selection (e.g. 0.2-1.2 ml volume per cavity in standard 96-cavity microtiter plates; 20 to 60 μl in 384-cavity microtiter plates). Owing to this volume limitation of the cavities, it is often necessary to enrich the viruses before screening. Phage titers of between 1×10¹⁰ to 1×10¹¹ pfu/ml (Plaque Forming Units) are thus normally achieved, for example, for lysates of the bacteriophage T7. It can, however, be necessary for successful screening to bring a total number of phages between 1×10¹⁰ to 1×10¹ pfu/ml or more into contact with the affinity ligand, and thus the concentration of the viruses from the lysate cannot be avoided before microtiter plate-based screening owing to the limited sample volume of the individual cavities (see above) (T7Select® System Manual, Novagen, Madison (USA) (TB178 06/00), page 6 et seq.). This normally occurs by means of a polyethylene glycol-mediated precipitation of the viruses from the culture supernatant and subsequent sedimentation of the precipitate. The disadvantage hereof is that the virus precipitates hereby forming can often only be re-dissolved with difficulty. The screening process is thus negatively influenced by the solubility behaviour of the viruses found in the precipitate. Furthermore, procedural steps connected with the precipitation of viruses are time-consuming and difficult to automate.

A method in which the culture supernatants/lysates resulting from the multiplication of the phage library can be directly used in the screening process is more advantageous. This is enabled by using incubation vessels having a larger sample volume than allowed by the cavities of microtiter plates.

It is further disadvantageous that a competing, simultaneous selection of structurally similar ligands against the phage library is ruled out by the separated volumes since only one ligand can be immobilised per cavity. A selection of the interaction partners occurring in a common sample volume, in which the immobilised interaction partners are arranged in a position-addressable, two-dimensional array, is advantageous. Suitable herefor are planar carriers (e.g. membranes) which are incubated in large-volume vessels (dishes or tubes) suitable therefor or which themselves form a vessel.

In order to achieve a uniform distribution of the phage population over the entire carrier surface, it is advantageous to ensure a uniform thorough mixing of the sample liquid. This can be accomplished, for example, by moving the vessel containing or forming the carrier by means of a tilting device.

Hawlish et al. (Analytical Biochemistry 293, 142-145 (2001)) describe a method for the selection of epitope-specific scFV fragments by means of an M13-based virus system. A peptide array synthesised on cellulose membranes was used for selection, which represents a part of the primary sequence of the human C3a receptor in the form of fifty 15mer peptides overlapping in the sequence. All viruses interacting with the array were eluted together and multiplied together following each selection round.

The identification of interacting viruses occurred in a separate bonding assay (ELISA) with the complete protein domain as the ligand. Part of the selected viruses was clonally isolated for this purpose after the fourth selection round, 92 clones were separately multiplied and analysed in the bonding assay. The increase in ELISA positive viruses as compared to ELISA negative viruses was evaluated as selection success. An assignment of ELISA positive viruses as bonding partners as compared to the individual affinity ligands of the array used for selection occurred by means of further ELISA-based assays. The entire array was brought into contact with an individual virus clone for this purpose and the assignment of the virus clone to one or more affinity ligands of the array was provided via the position of the bonding signal. A separate assay must thus be carried out for each virus clone against all affinity ligands contained in the array. The assignment of the virus clones to the individual affinity ligands immobilised in the array occurred in a further bonding assay (ELISA) carried out on the array.

The disadvantage of this method is that a common ELISA for identifying interacting virus clones is only possible when using peptidic affinity ligands derived from a common protein/polypeptide sequence. When using non-peptidic affinity ligands representing a combinatorial variety, a bonding assay must be carried out for each affinity ligand used in the array.

A disadvantage of using membranes as the surface is that the local concentration of the ligand can only be controlled with a lot of effort. This can lead to the formation of non-specific virus-ligand complexes owing to local avidity effects.

A very big disadvantage of the above method is that the spatial information of the array with regard to the ligands is lost during selection since the interacting viruses
i) cannot be detected on the array and
ii) cannot be eluted from the array in a site-specific manner.

In order to be able to use the spatial arrangement of the ligands in a two-dimensional grid to identify ligands interacting with viruses,
i) a measuring system is required, by means of which the bonding of the viruses during selection can be detected, and
ii) a method is required, by means of which bonded viruses can be eluted from the array in a site-specific manner.

In order to be able to detect the interactions of the viruses with the immobilised ligands during selection, it must be possible to carry out selection and detection of the selection success in the same measuring system. If marker-based detection methods (e.g. ELISA assays) are used, the bonding assay forming the basis for selection will be restricted during performance to the conditions of the marking reaction, and thus a variation of the bonding assay which is advantageous for selection, e.g. a change in the pH value, the ionic strength or the use of detergents, is not possible.

A big disadvantage of marker-based detection methods as used in the method cited above is furthermore that the viruses identified in the selection process cannot be used for the further method steps. If marker molecules (e.g. antibodies, streptavidin) are used, these require a physical interaction between the ligand-virus complexes and the marking reagent. This physical interaction can lead to a change in the bond between the ligand and the virus (weakening or strengthening) or even to impairment of the host-virus interaction (loss of infectiousness). It must be expected when using marker-based detection methods that insoluble aggregates form, and thus the viruses contained therein are no longer available for further method steps.

In order to be able to carry out selection and detection in the same measuring system and to be able to use identified viruses in the further method steps, a marker-free detection method (such as, for example, surface plasmon resonance (SPR)) must consequently be used. This also has the advantage that the direct bonding of the peptide or protein presented on the virus with the ligand can be detected.

A method in which bonding occurrences during the selection process are detected by means of a marker-free detection method, namely surface plasmon resonance (SPR), was described by Malmborg et al. (J. Immunol. Methods 198: 51-57 (1996)) for the selection of M13 virus-based antibody libraries against a ligand immobilised on a BIAcore™ biosensor. For this purpose, the proteins used as ligands (lysozyme, HM90-5, pB-1) were each covalently linked to the dextrane matrix of a sensor chip in three different samples, and a limited volume of a phage library was passed over the sensor surface in a continuous flow. The viruses were subsequently eluted from the surface with a solvent in a continuous flow and the eluate was collected in a time-fractionated manner. The progress of the selection carried out on the sensor surface was observed by means of time-resolved SPR measurement in a BIAcore™ device. The viruses contained in the eluate were separated and multiplied. The increase in the ratio of bonding to non-bonding of the virus clones contained in the eluate, which was detected by an ELISA, was deemed to be the primary selection success. The antibodies encoded by the interacting viruses were subsequently recombinantly produced and the dissociation constants thereof as compared to the ligand immobilised on the sensor chip were determined.

A very big disadvantage of this method is that a flow system is used. The arrangement of the sensor surfaces in a one-dimensional direction is thereby pre-determined (one-dimensional array). The disadvantage hereof is that it is precisely due to the use of a flow system that a two-dimensional sample arrangement (two-dimensional array) and the miniaturisation thereof is made considerably more difficult.

Competing affinity selection can furthermore only be laboriously realised in a flow system. The marker-free selection of a plurality of virus clones, which is, in the end, the result of a massively parallel screening set-up, can furthermore not be accomplished with the available technology.

The object of the invention is thus to provide a highly-parallelisable method for the selection and identification of peptide or protein molecules which can specifically interact with certain other molecules to form a bond, wherein the disadvantages of the prior art are avoided or reduced.

### SUMMARY OF THE INVENTION

The object according to the invention is solved by means of a method for the selection and identification of at least one representative (interaction partner) from a plurality of peptide or protein molecules, which can specifically interact with at least one representative from a plurality of molecules by forming a bond, said method comprising the steps of:
(a) bringing a virus system consisting of a plurality of viruses, each virus respectively presenting at least one representative from the plurality of peptide or protein molecules on its surface, into contact with the plurality of molecules (ligands) immobilised on the surface of a solid phase carrier such that they are position-addressable in a two-dimensional grid;
(b) removing unbound viruses from the surface; and
(c) identifying the interaction partner by detecting and determining the position of the bond between the immobilised ligand and the interaction partner presented by the virus with the aid of a marker-free detection method.

According to the invention, the ligands are immobilised in a two-dimensional array on a specifically designed solid phase carrier which enables a marker-free detection of interaction partners. The selection and detection of the selection success can thereby be carried out in one measuring system. The detected, interacting viruses can be further treated in successive method steps and can be multiplied if necessary, with either all the bonded viruses being used for this purpose or only those which have bonded to surface fields chosen for the respective selection. A further advantage of a marker-free detection method is that the direct bond between the ligand and the peptide or protein presented on the virus is detected. This is not the case when marker-based detection methods are used.

Since the plurality of ligands is immobilised in a two-dimensional grid, the method according to the invention furthermore enables, in connection with a suitable measuring system, a parallel detection with high integration density. A phage display method which is miniaturised and parallelised to a great extent is thereby provided, and thus detection can occur in parallel for several or all ligands.

It is furthermore advantageous that the culture supernatants/lysates resulting from the multiplication of the phage library can be directly used in the screening process and thus the time-consuming enrichment of the viruses from the culture supernatant/lysate is not necessary. It is also advantageous that the selection occurs in a common sample volume and thus a competing, simultaneous selection can be carried out against a plurality of ligands.

Steps (a) and (c) are preferably carried out on the same surface of the solid phase carrier, with the ligands being immobilised in a Cartesian grid (array) on the surface of the solid phase carrier, such that the position of any ligand can be determined by means of its x and y coordinates on the array. A plurality of position-addressable surface fields, also referred to according to the invention as ligand fields, can also be provided on the solid phase carrier on which the ligands are immobilised. The unbound viruses in step (a) are removed in step (b) preferably by means of elution.

In a preferred embodiment, the solid phase carrier contains a polymer-free surface on which the ligands are immobilised. Owing to the very high protein adsorption resistance of this polymer-free surface, it is possible to observe relatively weak interactions, i.e. bonding between the ligand and the protein or peptide molecule presented by the virus, which in particular allows the use of low-molecular-weight ligands.

The marker-free detection method is preferably based on an optical, electric or oscillation-based method. A reflection-optical method, in particular surface plasmon resonance (SPR), has proven to be particularly suitable. In this case, the solid phase carrier can be configured as an SPR sensor surface support.

Infection of the host cells for the multiplication of the viruses preferably occurs by means of the viruses bonded to the surface of the solid phase carrier. The advantage of this is that the bond between the ligand and the virus-presented peptide or protein does not have to be removed.

### BRIEF DESCRIPTION OF THE DRAWINGS

The method according to the invention can preferably be carried out in a device according to FIGS. 1 to 3.

FIGS. 1a to 1c show perspective views of SPR-sensor surface supports;

FIG. 2 shows a cross-sectional perspective view and an enlarged section of an SPR sensor surface support and a volume element;

FIG. 3 shows a sectional view and perspective view of a measuring area and the corresponding sealing member.

### DETAILED DESCRIPTION OF EMBODIMENTS OF THE INVENTION

The method according to the invention allows the selection and identification of one or more representatives of peptide or protein molecules from a plurality of such molecules. In this context “representative” means that each different peptide or protein molecule in the plurality of molecules does not normally occur as an individual molecule, but is rather present in the protein mixture to a greater or lesser extent. The selection and identification principle is then based on the fact that the peptide or protein molecule sought can interact with one or more previously chosen “selection molecules” to form a bond. These selection molecules are not particularly limited as regards their nature and can have any structure provided that they can be used at all in such a test and are able to form a bond. According to the invention they are therefore also simply referred to as “molecules”. The expression “ligand” is also used within the context of the present invention for those molecules that are immobilised on the surface of the solid phase carrier. A peptide or protein molecule which is capable of interaction, i.e. bonding to the ligands, and which can be selected and identified in this manner is also referred to as an “interaction partner”.

An enrichment, preferably an individualisation, of “interaction partners” is to be understood by the expressions “identification” and “selection” within the context of the present invention. This thus includes both the identification of interaction partners in a large variety or population of any different interaction partners and also the selection of individuals in a population enriched beforehand.

The interaction between the interaction partner and the ligand, which manifests itself in the form of a bond between the partners, can, for example, be characterised by a “lock and key principle”. The interaction partner (peptide or protein) and the selection molecule (ligand) have structures or motifs which are specifically compatible with each other, such as, for example, an antigenic determinant (epitope) which interacts with the antigen binding site of an antibody. From the knowledge of the structure of one of the binding partners conclusions regarding possible preferred structures or specific structural elements of a suitable partner interacting therewith can be drawn.

In the method and measuring system according to the invention, the interaction partners are presented on the surface of viruses as peptides or proteins. Hereby included are all peptides or proteins whose encoding nucleotide sequences can be inserted in a virus genome. It is preferred that the expression of these peptide or proteins as part of the virus shell allows the assembly of this shell and thus propagation of the virus. The propagated virus is preferably infectious.

The interaction partners can also be presented on the surface of cells, in particular also bacterial cells or spores, as peptides or proteins.

The expression peptides or proteins includes both natural and synthetic peptides or proteins. Examples of natural proteins include, inter alia, antibodies, antibody fragments, receptors that interact with their specific ligands, peptide ligands that interact with their specific receptors or peptide domains, that interact with specific substrates including proteins and coenzymes, and other peptides or enzymes etc. Also included herewith are recombinantly produced forms of the aforementioned proteins or peptides. Natural peptides correspondingly include, inter alia, fragments of the proteins described above, which interact with specific ligands. Synthetic proteins or peptides include both expressed pseudogenes or fragments thereof as well as proteins or peptides having a random amino acid sequence. The peptides and proteins are thus preferably components of a library consisting of viruses, with the viruses containing a nucleic acid sequence, preferably integrated in their genome, which encodes the corresponding peptide or protein. This nucleic acid sequence is thereby typically such that it leads, during expression, to the synthesis of the peptide or protein as a component of a fusion protein consisting of a coat protein of the virus or a part thereof and of the peptide or protein. This fusion protein is then able to be localised on the surface of the virus and is consequently able to present the peptide or protein.

Within the context of the method and measuring system according to the invention, the expression “ligand” describes molecules or compounds immobilised on the surface of a solid phase carrier. The expression includes macromolecules as well as “small organic molecules”. Furthermore, within the context of the present invention, general structural elements which, owing to their structural properties, can interact with peptides or proteins presented on viruses are referred to as ligands. From the knowledge of, the structure of the ligands conclusions regarding, inter alia, the possible structure or specific structural elements of the molecule presented on the virus can thus be drawn.

Molecules having a high molecular complexity or a high molecular weight are understood by the expression “macromolecules”. These are preferably biomolecules such as, for example, biopolymers, in particular proteins, oligopeptides or polypeptides, but are also DNA, RNA, oligonucleotides or polynucleotides, isoprenoids, lipids, carbohydrates (glycosides) as well as modifications thereof and also synthetic molecules. In connection with proteins, receptors in particular come into question, but also proteins or peptides that represent epitopes or antigenic determinants of proteins. The proteins can furthermore also be fusion proteins.

Molecules having a lower molecular complexity than the macromolecules described above are understood by the expression “small molecules” or “low molecular weight molecules”. The expression “small molecules” or “low molecular weight molecules” is not used uniformly in literature. In WO 89/03041 and WO 89/03042, molecules having molecular masses of up to 7000 g/mol are described as small molecules. However, molecular masses of between 50 and 3000 g/mol, more often of between 75 and 2000 g/mol and mostly in the range of between 100 and 1000 g/mol are usually specified. Examples are known to the person skilled in the art from documents WO 86/02736, WO 97/31269, U.S. Pat. No. 5,928,868, U.S. Pat. No. 5,242,902, U.S. Pat. No. 5,468,651, U.S. Pat. No. 5,547,853, U.S. Pat. No. 5,616,562, U.S. Pat. No. 5,641,690, U.S. Pat. No. 4,956,303 and U.S. Pat. No. 5,928,643.

Within the scope of the present invention, the expression “small organic molecules” will be used for molecules having a molecular weight of less than 3000 g/mol, preferably less than 1000 g/mol, most preferred less than 750 g/mol. Oligomers or small organic molecules such as oligopeptides, oligonucleotides, carbohydrates (glycosides), isoprenoids, lipid structures or haptens, can be cited as examples of such small molecules. In literature, most often the molecular weight represents the basis for the definition of such small organic molecules.

One aspect of the method according to the invention and the measuring system used relates to the provision of a two-dimensional array having a plurality of ligands on a solid phase carrier. The ligands are thereby arranged in the array such that the identity of any ligand can be determined by means of its x and y coordinates on the array.

The spatial structure of the resulting array can be predefined by means of a mechanical structuring of the carrier. If structured solid phase carriers are used in the present invention, they therefore preferably comprise a plurality of regularly arranged, position-addressable fields (ligand fields). These ligand fields contain one or more cavities (sensor fields) on the base of which the ligands are immobilised. The cavities preferably have a depth of 20 to 100 μm.

These ligand fields preferably each differ as regards the type of interaction partner immobilised on their sensor fields, with an individual ligand field being able to present both an individual ligand as well as several identical or different ligands. In a typical example, four interaction partners are immobilised per ligand field.

The cavities are preferably arranged such that a regular, preferably Cartesian grid of columns and rows occurs on the carrier. The size and shape of the carrier can be selected as desired and can easily be adapted to the detection system used. When using spotting robots to immobilise the ligands and/or when presenting the ligands in microtiter plates, the distance of the fields to one another is preferably to be adapted to the microtiter format used or to the format of the spotting device used. The number of fields on the solid phase carrier can also exceed the number of subunits of the microtiter plate, i.e. the density of the sensor fields on the solid phase carrier can be many times larger than the density of the subunits of the microtiter plate. Thus, a rectangular solid phase carrier can, for example, have 6144 fields which can be occupied by means of a spotting robot from four conventional 1536-cavity microtiter plates.

The method according to the invention is preferably carried out using an SPR sensor surface support as the solid phase carrier, which is divided into a plurality of measuring areas, with each measuring area comprising one or more (e.g. four) SPR sensor surfaces. At least one of these measuring areas is surrounded by an isolating area which does not contain any separating means, and is configured to accommodate a sealing member so as to form, together with a volume element to be placed on the measuring area, a space above this given measuring area, which is isolated from adjacent measuring areas.

This SPR sensor surface support enables a volume to be created above one or more measuring areas, which is isolated from adjacent measuring areas so that, for example, further measurements of samples disposed on the respective SPR sensor surfaces can be carried out directly on the SPR sensor surface support.

It is thus possible to place a volume element on the SPR sensor surface support such that respective volumes are created above one or more measuring areas, which are large enough for further desired measurements and analyses to be carried out. This creates the significant advantage that such further measuring and analysis steps can be carried out directly on the SPR sensor surface support, which greatly simplifies the performance of the measurements and reduces the total equipment outlay since it is not necessary to provide separate measuring volumes.

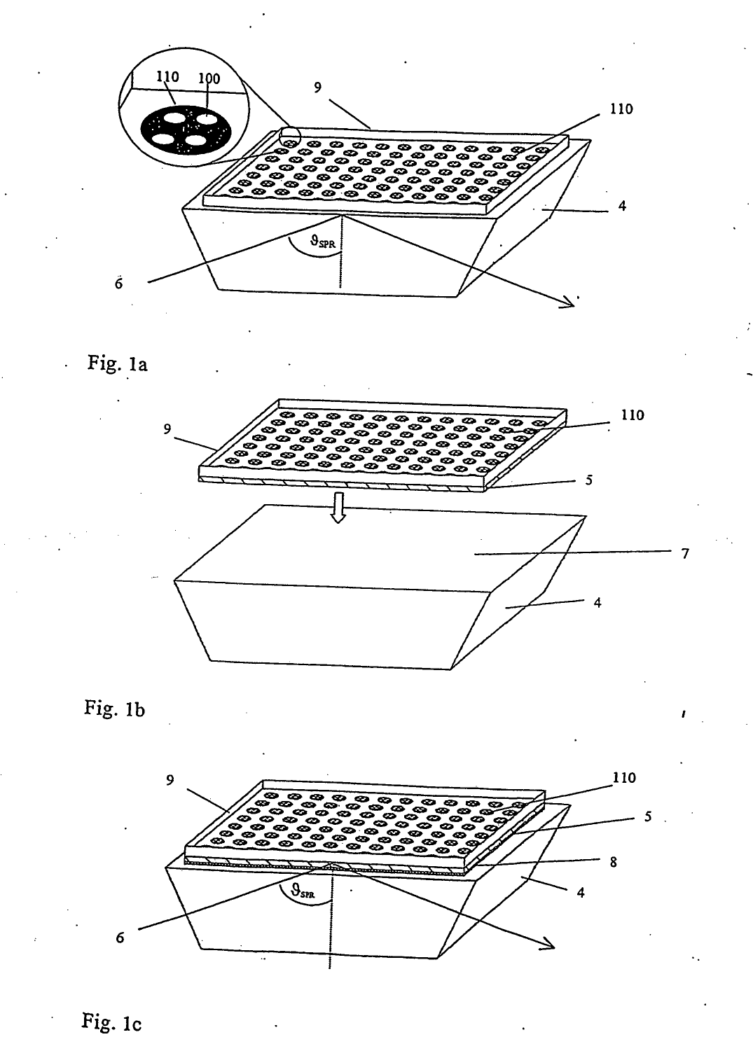

Shown in FIG. 1a is a first design of the SPR sensor surface support, in which a plurality of measuring areas 110, each comprising four SPR sensor surfaces 100 in the shown example, are arranged on a prism 4 which serves in this example as the substrate of the SPR sensor surface support. Also shown is a cuvette bordering 9 that is preferably placed around the entire arrangement of) measuring areas 110. Also shown is a light beam 6 which is passed through the prism 4 (the SPR sensor surface support) so as to excite a surface plasmon resonance in the SPR sensor surfaces 100.

FIG. 1b shows a further design of the SPR sensor surface support, in which a plate-like substrate 5 supports the SPR sensor surfaces 100 and the measuring areas 110. A cuvette bordering 9 is also provided, similar to the design of FIG. 1a. In order to carry out a measurement with this SPR sensor surface support, it is placed on a surface 7 of a prism 4 such that light (more generally speaking: SPR exciting radiation) 6 can be passed through the prism 4 and the plate 5 to the SPR sensor surfaces 100. This preferably occurs by using an index liquid 8 between the prism 4 and the plate 5 so that the light 6 shone in under SPR conditions is not reflected on the interface of the surface 7 at the air gap in front of the plate 5. Thus, the light penetrates through the index liquid 8 into the light-transmitting sample carrier 5 thereabove and is reflected only on the side of the SPR sensor surfaces 100 that is coated with SPR-compatible material. An example of an index liquid is oleic acid or an oleic acid-containing mixture.

Any material transparent to SPR-compatible radiation, on which an SPR-compatible material can be applied in the SPR sensor surfaces 100, comes into question as the material for the prism 4 and the plate 5. For example, the substrate 4 or 5 may consist of glass, and the SPR sensor surfaces may be formed by a metal coating, in particular by a gold layer.

As is shown in FIGS. 1a to 1c, it is preferred for the measuring areas 110 to be addressable in two dimensions. In this context, the expression “addressable” means that individual measuring areas can be distinguished from each other by means of a corresponding identification or address, with it accordingly also being possible to address samples correlated therewith. This creates the advantage that a very large number of measuring areas 110 can be simultaneously exposed to light and evaluated. It is, however, also possible within the scope of the invention to arrange the measuring areas so as to be addressable in one dimension.

It is particularly preferred for the measuring areas 110 to be arranged in a Cartesian grid, as shown in FIG. 1, with the addressability then being given most easily by Cartesian coordinates. The present invention is, however, in no way limited to this, and the measuring areas can be distributed in a random grid or also in an completely disorderly manner, and can be addressed regardless of their specific arrangement according to any coordinates (e.g. according to polar coordinates).

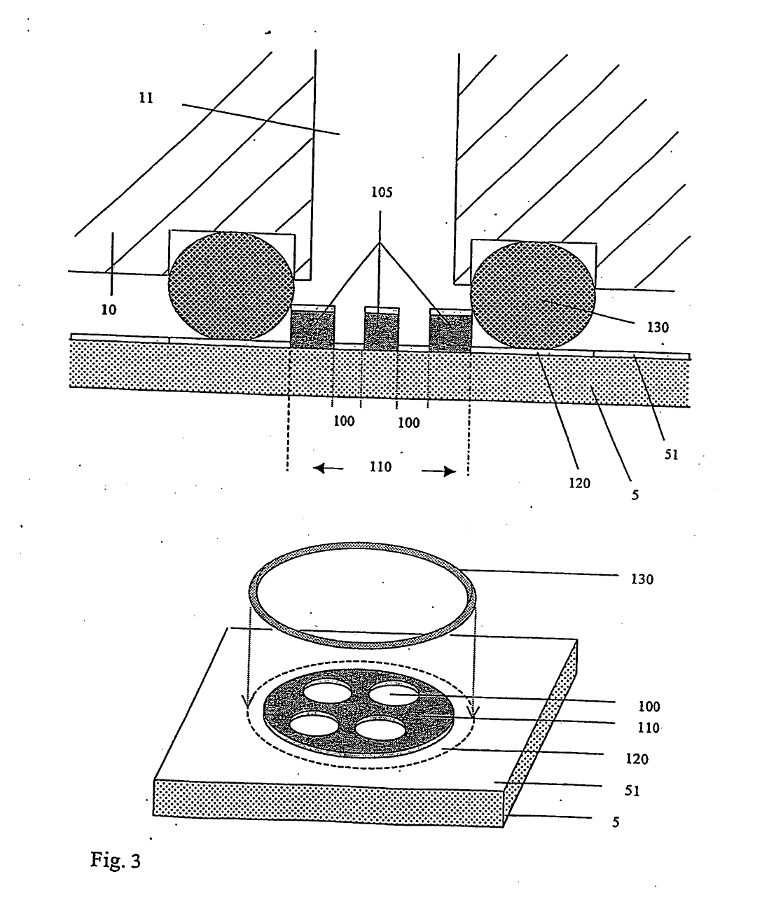

The structure of an individual measuring area 110, given by way of example, and of a corresponding isolating area 120 will now be described in more detail with reference to FIG. 3. FIG. 3, bottom, schematically shows the carrier 5 which has a gold layer 51 disposed thereon. For the sake of simplicity, only one measuring area 110 is depicted. The measuring area 110 comprises four SPR sensor surfaces 100 in the shown example. It should, however, be noted that a measuring area may also comprise more or less SPR sensor surfaces 100. The measuring area 110 is formed by suitable separating means 105 (examples of which will be described later) which are mounted on the carrier 5, as is shown in the sectional view at the top of FIG. 3, and which separate the gold layer 51 from the carrier such that SPR sensor surfaces 100 are formed, in which the gold layer is applied on the carrier 5 (possibly with a layer therebetween for promoting adherence between the gold layer 51 and the carrier 5), and separating means areas in which the separating means 105 are mounted on the carrier or substrate 5 (possibly likewise with an adherence-promoting intermediate layer). Thus, when light impinges from below through the carrier 5 to the top surface, a surface resonance may occur in the SPR sensor surfaces 100 under suitable angle and wavelength conditions of the incident SPR radiation, whereas the separating means are configured such that no surface resonance occurs there, the SPR sensor surfaces thus being clearly separated from each other by the structuring of the surface of the carrier 5.

The shown measuring area 110 is preferably surrounded by an isolating area 120. The isolating area 120 is configured to accommodate a sealing member 130 so as to form, together with a volume element 11 to be placed on the shown measuring area 110 (see FIG. 3, top), an isolated space above this measuring area.

It can be seen that the isolating area 120 does not include any separating means 105. It is thus ensured that reliable sealing can be achieved by the sealing member 130.

The isolating area 120 is preferably configured on the surface facing away from the substrate 5 in the same manner as the SPR sensor surfaces. This can be seen in FIG. 3, top, since both the SPR sensor surface and the isolating area 120 have the gold surface 51. According to a preferred embodiment, not only are the surfaces configured in the same manner, but the SPR sensor surfaces and the isolating areas are also identical overall, i.e. they have the same layer sequence from the substrate 5 to the surface. In other words, the SPR sensor surfaces 100 and the isolating areas 120 are preferably produced using the same method steps, and thus no separate method steps are required for their respective production.

It is, however, also possible for the isolating area 120 to be configured on the surface facing away from the substrate 5 in a different manner to the SPR sensor surfaces 100. An isolating area 120 can, for example, be formed by an unoccupied substrate surface. It is possible, as a further alternative, for an isolating area 120 to comprise a seal-enhancing layer on its surface, which consists of a material matching the sealing members 130, e.g. silicon. This seal-enhancing layer can be applied in any desired or practical manner such as, for example, by means of a mask, by means of a robot which controls the individual isolating areas, or even manually.

According to a preferred design, the separating means 105 are raised not only as compared to the SPR sensor areas so as to thus form the respective cavities on the SPR sensor surfaces, but also as compared to the isolating area 120 as shown in FIG. 3. It is thus possible, if the isolating area 120 and the sealing member 130 are suitably sized, for the separating means 105, which form the circumference of the measuring area 110, to serve as a guide for the sealing member 130. This facilitates placement of the sealing members 130.

As is shown in FIG. 3, the measuring areas 110 and the corresponding sealing members 130 are preferably configured so as to be round or oval. It should, however, be noted that the invention is applicable to any geometric shapes of the outer circumference of measuring areas and sealing members.

FIG. 3 shows an individual measuring area 110 with the corresponding isolating area 120. Although it is fundamentally possible to provide a plurality of measuring areas (as shown in FIG. 1), with only one or a few of these measuring areas being surrounded by corresponding isolating areas, it is preferred for each of the plurality of measuring areas 110 of a given SPR sensor surface support to be surrounded by a corresponding isolating area 120. This results in the advantage that it is possible, by placing a corresponding sealing member and volume element on top, to form a volume above any measuring area 110 to carry out further measurements and analyses.

A method for producing an SPR sensor surface support according to the designs as described above will now be set forth. This preferably occurs by forming or applying the separating means 105 to the respective substrate, e.g. the plate 5 or the prism 4, such that free areas are created between the separating means 105, which define SPR sensor surfaces 110 and isolating areas 120, and by then applying an SPR-compatible material at least in the free areas which define SPR sensor surfaces 100.

If the SPR-compatible material (e.g. gold) is applied only in the free areas which define SPR sensor surfaces, an SPR sensor surface support is formed in which the isolating areas are characterised by the unoccupied substrate or the layer directly below the gold layer. If the SPR-compatible material is also applied in the free areas which define isolating areas, an SPR sensor surface support as shown in FIG. 3 is formed, namely in which the SPR-compatible layer is present in both the SPR sensor surfaces 100 and in the isolating areas 120.

A seal-enhancing layer (e.g. silicon) can be applied to the isolating areas as a supplementary step regardless of whether or not the SPR-compatible material was applied to the isolating areas.

The step of forming the separating means 105 can be carried out, for example, by applying a polymer to the surface of the substrate 4 or 5. This preferably comprises the steps of applying a photostructurable polymer to the entire surface of the substrate 4 or 5, exposing the applied polymer layer to light using a mask which defines areas belonging to the separating means 105, areas belonging to the SPR sensor surfaces 100 and areas belonging to the isolating areas 120, and processing the exposed polymer layer so as to vacate the substrate surface in the areas belonging to the SPR sensor surfaces 100 and the isolating areas 120.

An alternative when applying a polymer for the separating means is the application of a polymer to the surface of the substrate 4 or 5 in a two-dimensional grid which defines the separating means 105, the SPR sensor surfaces 100 and the isolating areas 120, and curing the polymer. In this alternative, the polymer is preferably applied by means of a screen printing technology.

As an alternative to forming the separating means using a polymer, the separating means may also be formed from a structurable silicon layer.

In all of the production methods set forth above, the step of applying the SPR-compatible material preferably occurs by means of the deposition of a metal, with it being possible to apply an adherence-promoting layer before deposition of the metal. It is particularly preferred for the metal to be vapour-deposited onto the entire surface of the structured substrate so that the separating means are then also covered, as is schematically illustrated at the top of FIG. 3.

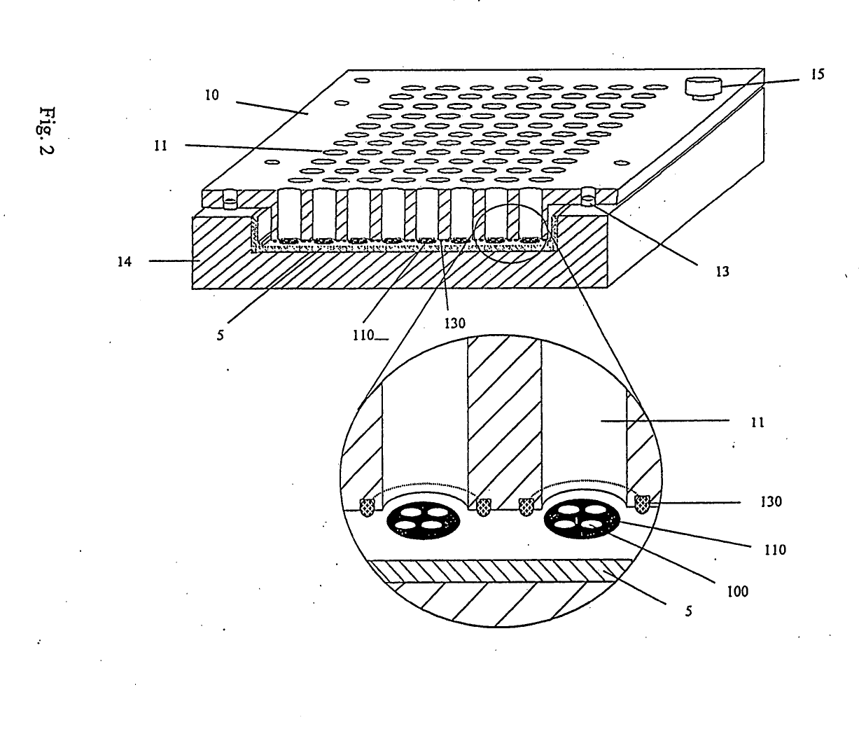

As already described above, the present SPR sensor surface support is configured such that the isolating area 120 may accommodate a sealing member 130 to form, together with a volume element 11, an isolated space above the measuring area. The volume element 11 can be provided in any suitable or desired manner, e.g. as a cylindrical single element that is connected to a single sealing element 130. Several volume elements 11 are, however, preferably provided as part of a volume element carrier 10, as shown, for example, in FIG. 2. To be more precise, FIG. 2 shows measuring means consisting of an SPR sensor surface support 5 and a volume element carrier 10, which interact with one another such that spaces are formed above the respective measuring areas 1 10.

Although it is possible for the individual volume elements to be detachable components of a volume element carrier 10, it is preferred for the volume element carrier 10 to be a body in which the volume elements 11 are formed as bores or recesses. The volume element carrier 10 may be produced, for example, by material-removing machining (e.g. milling or drilling), from a plastic (e.g. Teflon) or metal (e.g. aluminium). Thermoplastics (e.g. polystyrene or polypropylene) come into question as alternative plastics, even though they are less suitable for material-removing machining than, for example, metallic materials. In addition to the material-removing machining of a material block, it is also possible to bring the volume element carrier into the desired shape by means of a casting method (e.g. injection moulding). When using injection moulding, any shapable or solidifying materials suitable herefor, e.g. the aforementioned thermoplastic elastomers such as polystyrene or polypropylene, or also castable metals, can be used.

If the volume element carrier is to be used repeatedly in methods in which viruses, bacteria or other potentially infectious biological entities are used, materials are preferably selected that are resistant to chemical sterilisation (e.g. treatment with citric acid, NaOH/SDS). Such a material is, for example, PolyChloroTriFluoroEthylene (PCTFE).

As shown in FIG. 2 and as is also indicated at the top of FIG. 3, the sealing members 130 are preferably components of the volume element carrier 10. The sealing members 130 can thereby be fixedly or releasably connected to the volume element carrier 10. Grooves are preferably provided on the side of the body forming the volume element carrier, around the openings defining the volume elements 11, in which the sealing members 130 are placed. In this case, the sealing members are preferably O-rings.

It is, however, also possible for the sealing members and the volume element carrier to be integrally formed. This is possible, for example, if the volume element carrier is produced by means of injection moulding from a suitable plastic material that is sufficiently flexible for the sealing members. In this case, the sealing members may be formed as protruding beads shaped so as to fit the measuring areas (e.g. as ring-shaped beads for round or oval measuring areas) on the side of the volume element carrier that is to be placed onto the SPR sensor surface support.

In general, seals of soft materials made, for example, of plastic, rubber, silicon, Teflon or the like, which can be used in a ring, lamella or mat configuration are suitable as sealing members. Vacuum seals can also be used.

The SPR sensor surface support and the volume element carrier 10 preferably have respective adjustment members, e.g. set pins and guides 13 (see FIG. 2), to ensure that the sealing members 130 can be aligned with the corresponding isolating areas 120. The tolerances for the set pins and guides are sized to fit the dimensions of the measuring areas and/or isolating areas and the sealing members so that the desired fitting accuracy between the sealing members and the isolating areas can be achieved. The fitting accuracy of the set pins and guides is preferably in the order of 20 μm or less.

It is moreover preferred for the SPR sensor surface support and the volume element carrier to comprise respective attachment members 15 to tightly connect the SPR sensor surface support and the volume element carrier to each other. The attachment members 15 are preferably such that the connection is also releasable. The connection members 15 can be, for example, a pressure connection such as a screw or clamp connection. In other words, the attachment members can be, for example, guides into which a metal clamp is inserted so as to connect the SPR sensor surface support and the volume element carrier with each other. Alternatively, the connection members can be bores with internal threads, into which an exterior screw is screwed to connect the SPR sensor surface support and the volume element carrier with each other.

It is also possible for the adjustment members 13 and the attachment members 15 to be identical, which would be possible, for instance, for the aforementioned example of threaded bores since screwing in the exterior screw on the one hand connects the SPR sensor surface support and the volume element carrier and on the other causes an adjustment due to the alignment of the bores. It should be noted, however, that the tolerances must establish the required fitting accuracy. It is therefore preferred for the adjustment members 13 and the attachment members 15 to be separate from each other since the requirements on the tolerances of the attachment members can then be lower.

In order to be able to increase the screening throughput, it is advantageous to immobilise as many representatives of ligands as possible. It is possible hereby to immobilise a number of at least 10, preferably 96,

particularly preferred 384, especially preferred 1536, more particularly preferred 4608, most particularly preferred 9216 different representatives of ligands. Within the scope of the present invention, it is also possible to immobilise identical ligands several times. This can be useful, for example, in the case of multiple determinations of the ligand-virus interaction in order to evaluate the quality of the selection process. The given figures thus only take into account ligands differing from each other. It must also be taken into consideration that different sites of the ligands can be used to immobilise the ligands. Thus, the ligand has a different orientation on the surface and may sometimes exhibit different bonding behaviour. In accordance with the invention, different orientations of a ligand are also understood as “different ligands” to simplify matters.

The structuring of the sensor fields occurs by means of separating means as described in the PCT patent specification WO 01/63256, and reference is herewith made to the entire disclosed content of this document.

If surface plasmon resonance (SPR) is used to detect interactions, an inexpensive structuring of the sensor fields can be achieved by not using the sensor itself (preferably a prism) as the solid phase carrier and directly structuring this, but by instead using a separate sample carrier as the solid phase carrier for immobilising the ligand, which is placed onto the sensor.

The solid phase carrier can consist, for example, of glass, plastic or metal, preferably a precious metal, particularly preferred of gold, and has a layer of such a metal on the surface. This metal layer can be applied, where necessary, with the help of an intermediate layer which serves to promote adhesion. The material used, on which the surface is applied, is dependant on the measuring method used. The solid phase carrier is preferably suitable for the use of marker-free detection methods.

It is advantageous particularly when using reflection-optical methods for the solid phase carrier to have at least two parts (see above), with it being possible for one part to be made of an at least partially light-transmitting material and the other to be a metal layer. This can be in particular a glass body and a gold layer. A prism or glass plate, for example, comes into question as the glass body. The carrier is preferably suitable for surface plasmon resonance (SPR). A suitable sensor and measuring system is disclosed, for example, in WO 01/63256, and reference is made herewith to the entire disclosed content of this document. The immobilisation of the ligand can occur directly or indirectly on the solid phase carrier. There are several possibilities of binding ligands to a solid surface. Covalent, ionic or adsorptive bonding can be cited here as examples. Covalent bonding of the ligand to the carrier is particularly preferred since this chemical bond is so stable that it allows adhering proteins to be completely denatured without affecting the surface properties.

The ligand can be used unaltered or chemically modified. Chemical modification includes the alteration of existing reactivities such as activating existing functional groups or adding a further molecule which enables direct or indirect linkage to the surface. Simple addition or substitution reactions can be used herefor.

An organic intermediate layer is advantageous to prevent or reduce an undesired, frequently occurring non-specific binding of the ligand to the carrier surface, especially if the carrier consists of a plastic or metal surface. A self-assembling monolayer (SAM) is frequently used here, which prevents an adsorption of the ligand on the carrier. The self-organisation into a thick film normally occurs by means of a hydrophobic interaction between long-chain hydrocarbons having a functional group on their one end which enables the binding to the carrier, and containing a functional group on their other end which enables the binding of the ligand. Compounds comprising these functional components (head group, tail group, hydrophobic part) are also called anchors. The anchor may further have a spacer part containing preferably ethylene glycol units which ensure a low non-specific protein adsorption.

So-called diluent molecules are advantageously admixed to the aforementioned anchor molecules to control the concentration on the surface. Too dense a surface concentration can be disadvantageous owing to steric hindrance. Diluent molecules are structurally adapted to the anchor molecules, however, they do not have a head group for the binding of the ligand since this is supposed to be avoided. They are furthermore usually shorter than the anchor molecules in order to avoid impairment of the accessibility of the ligand for the peptide or protein presented on the virus.

In the prior art, a polymer such as, for example, dextrane is often additionally applied to the organic intermediate layer. Owing to the possible undesired interaction between this polymer and the ligand a polymer-free surface is preferred. A further advantage of a polymer-free surface is that the use of blocking reagents during the selection process can be dispensed with owing to the low non-specific protein bonding. This is particularly advantageous since these blocking reagents likewise exhibit non-specific protein bonding which is thus avoided. A further advantage of polymer-free surfaces is that they can be regenerated very easily. Reagents enabling regeneration of the surface in a one-step method (e.g. SDS-containing solutions or methanol-trifluoroacetic acid mixtures) can be used herefor.

One-step methods cannot be used for regeneration of the polymer surfaces used in the prior art since the reagents used thereby alter or destroy the structure of the polymers, or lead to the separation of the polymers from the carrier. On a polymer-free surface on the other hand, the selection of the regeneration agents is only limited as regards the stability of the ligand.

SAMs can be produced, for example, by chemisorption of alkylthiols on a metal surface (e.g. gold). The long-chain molecules pack together as SAMs on the solid phase, with the gold atoms being complexed by the sulphur functions. A further example is the silanisation of glass or silicon with reactive epoxide or amino group-containing silanes, and the subsequent acylation of the amino groups, for example by means of nucleoside derivatives (Maskos and Southern, Nucl. Acids Res. 20(1992)1679-84).

As regards the synthesis of anchors and diluent molecules, reference is made to WO 00/73796 A2 and to DE 100 27 397.1, and reference is herewith made to the entire disclosed content of the documents.

The application of the ligands to be immobilised is not limited to specific methods. To localise the active sites on the surface more precisely, conventional pipetting or spotting devices, but also stamping or inkjet methods can, for example, be applied.

The removal of the non-interacting viruses according to step (b) of the method according to the invention can occur according to conventional methods known to the person skilled in the art. The non-interacting viruses are preferably removed from the surface by means of elution. In accordance with the “interaction” as defined above, the expression “non-interacting viruses” comprises those viruses which do not interact with the immobilised affinity ligand(s), i.e. do not form a bond with the ligand. An elution method is, for example, a washing method. The surface can hereby be treated, for example, with suitable solutions, the composition of which ensures that the interaction between the interaction partner and the target molecule is not disrupted. Also included in this context are elution conditions of different stringencies, in which, for example, low-affinity interactions are disrupted and an enrichment or identification of high-affinity interaction partners thus occurs. Such examples are known from the prior art, for example in T7Select® System Manual, Novagen, Madison (USA) (TB178 06/00), page 14 et seq.

The detection of the interaction between the ligands and the interaction partners presented by the viruses according to step (c) of the method in accordance with the invention can take place according to conventional detection methods known to the person skilled in the art, in which it is ensured that viruses detected during the selection process can be used in the further method steps. This is the case when using marker-free detection methods.

In a preferred embodiment of the invention, the marker-free detection of the interaction between the ligands and the interaction partners presented by the viruses in step (c) is based on an optical, oscillation-based or electric method. It is particularly preferred for detection of the interaction to occur in a reflection-optical manner. The interaction is hereby preferably detected by determining the surface plasmon resonance (SPR).

It is very advantageous that owing to the use of a marker-free detection method, the same surface can be used for both the selection process and for the detection of bonding occurrences, which only requires a single surface evaluation and furthermore enables an identical ligand presentation.

In order to detect interactions between the ligands immobilised on the sensor fields and the interaction partners presented on the viruses by means of SPR, the sensor fields are imaged onto a position-sensitive detector. It is thereby possible to use every single sensor field as a separate measuring surface, i.e. the bonding of the phage particles can be detected separately for each sensor field. The detector should be capable of detecting all bonding occurrences in parallel, and detection itself should occur in parallel. The detector is advantageously a CCD camera. The advantage of parallel selection is that it promotes the comparability of the individual measuring results.

So that the sensor fields are visible with good contrast on the detected image, the light arriving in the intermediate regions of the ligand fields should be absorbed, scattered away or diverted away in a direction other than the direction of detection to the greatest possible extent. It is only this contrast between the sensor field and edging which allows an assignment of the pixel regions in the image to a sensor field to be defined. A summation is made, during data acquisition, over the pixels of a region in the image, and thus the spectra for the sensor fields also become more meaningful when there is good absorption of the intermediate regions.

Adjusting the system is easily possible since the sensor array (with or without samples on the sensor fields) is initially placed into the measuring system and imaging is then made by means of radiation under random radiation conditions, with the contrast permitting distinction between the individual sensor fields or between the sensor fields and the separating means. Various possibilities of eliminating light at undesired locations by means of separating means, and a measuring system for parallel detection are described in the PCT patent specification WO 01/63256, and reference is herewith made to the entire disclosed content thereof.

Detection step (c) can be followed by a further treatment step (d) which may comprise one of the following steps:
(d1) elution of all bound viruses;
(d2) elution of those viruses which are bound to ligands of selected surface fields;
(d3) addition of host cells to the entire surface;
(d4) addition of host cells to selected surface fields.

In step (d1), the interacting viruses are eluted from the surface and are added to a host cell culture for infection.

In step (d3), host cells are added to the entire surface and are infected by the interacting viruses, followed by elution of the infected host cells from the surface. The advantage of infection on the surface is that the ligand-virus complexes do not have to be dissolved.

In steps (d2) and (d4), only those viruses which have interacted with ligands immobilised on specific, selected sensor fields are further treated as described above. This is preferably achieved by means of a specifically designed grid mask that is applied to the ligand field containing the interacting ligand(s). The recesses of the grid mask are aligned in the same two-dimensional grid as the ligand fields on the carrier. The alignment of the two grids is achieved by means of an adjustment means (e.g. set pins) in the grid mask and the carrier support.

In step (d2), an eluent is added to those recesses of the grid mask which surround ligand fields containing interaction partners which interact with viruses on their sensor fields, followed by the elution of the interacting viruses from the surface.

In step (d4), host cells are added to those recesses of the grid mask which contain sensor fields with which viruses have interacted, followed by the elution of the infected host cells from the surface.

In a preferred embodiment of the invention, the interacting viruses or infected host cells of several recesses are multiplied together in step (d2) or (d4).

In a preferred embodiment, the method according to the invention further comprises a multiplication step (e) which is carried out following step (d):
(e) multiplication of the interacting virus by infection of a host.

The multiplication of the viruses occurs by diluting the infected cells in a preculture of the host strain and by subsequent growth of the culture until lysis occurs. The conditions for multiplying the interacting viruses by infecting a host are known to the person skilled in the art from the prior art, e.g. in T7Select® System Manual, Novagen, Madison (USA) (TB178 06/00), page 18 et seq.

It is furthermore preferred for the sequence of steps (a), (b) to be repeated one or more times following step (b) before the detection step (c) is carried out. It is particularly preferred for the further treatment and multiplication steps to be carried out one or more times, i.e. the sequence of steps (d), (e), (a), (b) is repeated one or more times, following step (b) before the detection step (c) is carried out. To verify that bonding has actually occurred and to select the corresponding ligand fields, step (c) may be carried out before step (d). Due to the repetition of these sequences of steps, a selective enrichment of viruses which present the interaction partners of the immobilised ligands on their surface is ensured.

In another preferred embodiment, the method further comprises a characterisation step (f) which is carried out either following step (c) or following step (e):
(f) characterisation of the bonding of the selected virus populations as well as of individual virus clones stemming from these virus populations to the ligand used for the selection in an assay.

Any kind of assay which is suitable for characterising a bonding comes into question here. Such an assay is preferably a solid phase assay. Methods known in literature are, for example, ELISA (Enzyme-Linked Immunosorbent Assays), RIA (Radioimmuno-Assay) as well as surface plasmon resonance (SPR) or oscillation resonance methods (Butler, J.E., METHODS 22, 4-23 (2000)).

In a preferred embodiment, characterisation of the bonding in step (f) occurs on the same or identical surface on which the virus population as well as the individual virus clones stemming from this virus population were identified and selected.

In addition, a preferred embodiment of the method further comprises the isolation and sequencing step (g):
(g) isolation and sequencing of the DNA segment of individual virus clones which encodes the peptide or protein of the interaction partner.

Suitable methods are known to the person skilled in the art from the prior art, which enable him to isolate and analyse, following the isolation of individual virus clones, the DNA sequences inserted therein which encode the corresponding peptides or proteins.

In a further preferred embodiment of the invention, the method also comprises the recombinant expression and isolation or the chemical synthesis of the peptide or protein identified/selected as the interaction partner of the ligand.

Different expression systems and thus different methods for the expression of isolated nucleic acid sequences and for the isolation of encoded peptides and proteins are known to the person skilled in the art from the prior art. These expression systems include procaryotic and eucaryotic systems (see in this regard, inter alia, chapter 9.4 “Expressionssysteme”, in: Mühlhardt, Der Experimentator: Molekularbiologie, Gustav Fischer Verlag 1999).

In another preferred embodiment, the method according to the invention further comprises the characterisation of the bonding of the recombinantly expressed or chemically synthesised peptide or protein with regard to individual ligands based on the selection of the ligands initially used in an assay. Owing to the comparability of the results, the same assays are advantageously used thereby as are employed when reviewing the virus clones. This is useful in order to detect a bonding of the selected interaction partner which is not dependent on the virus.

In a further preferred embodiment of this method, this characterisation occurs on the same surface as was used for the identification/selection of the corresponding virus.

In a preferred embodiment of the method and measuring system, the interaction partners presented by the viruses are encoded by DNA fragments inserted in the virus genome which form a DNA library. The DNA library thereby contains at least 10², preferably 10³, more preferred 10⁴, particularly preferred 10⁵, especially preferred 10⁶ and most particularly preferred 10⁷ DNA fragments.

In another preferred embodiment, the inserted DNA fragments are isolated from cDNA or genomic DNA (gDNA) or are synthetic oligo- or polynucleotides. It is furthermore preferred that the inserted cDNA or inserted gDNA stem from a procaryotic or eucaryotic organism.

The eucaryotic organism is thereby preferably a fungus, a plant or an animal organism, preferably a mammal. The mammal is preferably a mouse, a rat or a human being.

In another preferred embodiment, the cDNA is isolated from a differentiated tissue or a differentiated cell population. The isolation of cDNA from liver, brain, heart or breast tissue or cells is thereby preferred. The tissues or cells preferably stem from a healthy organism.

In an alternatively preferred embodiment, the tissues or cells stem from a diseased organism. The disease or ailment of the organism is preferably selected from the group consisting of cancer, hypertrophy and inflammation.

The viruses forming the virus system may comprise wild-type viruses and genetically modified viruses. In a preferred embodiment of the method, the virus system comprises a virus which uses eucaryotes as hosts. In another preferred embodiment, the virus system comprises a virus which uses procaryotes as hosts. The virus may have single-stranded DNA (ssDNA viruses) or may preferably be selected from the group of viruses having double-stranded DNA (dsDNA viruses). It is furthermore preferred for this dsDNA virus to be selected from the group consisting of bacteriophages. It is moreover preferred for the bacteriophage to be selected from the group consisting of tailed bacteriophages, more preferably selected from the group consisting of myoviridae, podoviridae or syphoviridae.

In another preferred embodiment, the bacteriophage is an Escherchia coli-specific bacteriophage.

The bacteriophage may also be a filamentous bacteriophage and is preferably selected from M13, fl and fd phages.

Also preferred is a virus system comprising a lytic phage. This lytic phage preferably has a polyhedral, in particular an icosahedral capsid. In a further preferred embodiment, the lytic phage is a λ phage, a T3 phage, a T4 phage or a T7 phage.

The method according to the invention can be used, for example, for epitope mapping or for the identification of peptide lead structures. Furthermore, the method according to the invention is an ideal method for identifying ligands which make the purification steps more efficient.

Particularly preferred is the use of the method according to the invention for identifying small organic molecules which interact with target proteins or target peptides from the human proteome (proteome mapping). The information hereby generated can be advantageously used for developing “small-molecule” active substances.

1. Method for selection and identification of at least one representative (interaction partner) from a plurality of peptide or protein molecules, which can specifically interact with at least one representative from a plurality of molecules by forming a bond, said method comprising the steps of:
(a) bringing a virus system comprising a plurality of viruses, each virus respectively presenting at least one representative from the plurality of peptide or protein molecules on its surface, into contact with the plurality of molecules (ligands) immobilised on a surface of a solid phase carrier such that they are position-addressable in a two-dimensional grid;
(b) removing unbound viruses from the surface; and
(c) identifying the interaction partner by detecting and determining the position of the bond between the immobilised ligand and the interaction partner presented by the virus with an aid of a marker-free detection method.

2. Method according to claim 1, wherein steps (a) and (c) are carried out on the same surface of the solid phase carrier.

3. Method according to claim 1, wherein the ligands are immobilised in a Cartesian grid (array) on the surface of the solid phase carrier, the position of any ligand can be determined by means of its x and y coordinates on the array.

4. Method according to claims 1, wherein the ligands are immobilised on a plurality of regularly-arranged, position-addressable surface fields (ligand fields).

5. Method according to claim 1, wherein the marker-free detection method is based on an optical, electrical or oscillation-based method.

6. Method according to claim 5, wherein the marker-free detection method is a reflection-optical method.

7. Method according to claim 6, wherein surface plasmon resonance (SPR) is used as the reflection-optical method.

8. Method according to claim 1, wherein detection occurs in parallel at least for several ligands.

9. Method according to claim 1, wherein the unbound viruses are removed from the surface in step (b) by means of elution.

10. Method according to claim 1, wherein the detection step (c) is followed by a treatment step (d) selected from the steps of:
(d1) elution of all bound viruses;
(d2) elution of those viruses which are bound to ligands of selected surface fields;
(d3) addition of host cells to the entire surface; and
(d4) addition of host cells to selected surface fields.

11. Method according to claim 10, wherein a step (e) follows the treatment step (d):
(e) multiplication of the viruses.

12. Method according to claim 1, wherein the sequence of steps (a), (b) is repeated one or more times following step (b) before the detection step (c) is carried out.

13. Method according to claim 11, wherein, following the step (b) a sequence of steps (d), (e), (a), (b) is repeated one or more times before the detection step (c) is carried out.

14. Method according to claim 13, wherein the step (c) is carried out before step (d).

15. Method according to claim 1, wherein macromolecules are used as molecules.

16. Method according to claim 15, wherein the macromolecules are selected from proteins, peptides, oligonucleotides, carbohydrates (glycosides), isoprenoids and lipids.

17. Method according to claim 1, wherein small organic molecules are used as molecules.

18. Method according to claim 15, wherein the molecules have a molecular mass of less than 3000 g/mol.

19. Method according to claim 18, wherein the molecules have a molecular mass of less than 1000 g/mol.

20. Method according to claim 19, wherein the molecules have a molecular mass of less than 750 g/mol.

21. Method according to claim 1, wherein at least Y different representatives of ligands are immobilized, with Y being selected from 96, 384, 1536, 4608, 6144 and 9216.

22. Method according to claim 10, wherein a step (f) follows the detection step (c):
(f) characterization of bonding between the ligand and interaction pat tier in an assay.

23. Method according to claim 11, wherein a step (f) follows the multiplication step (e):
(f) characterization of the bonding between the ligand and interaction pat tier in an assay.

24. Method according to claim 22, wherein the characterization step (f) occurs on the same surface on which the interaction partners were selected and identified.

25. Method according to claim 23, wherein the characterization step (f) occurs on the same surface on which the interaction partners were selected and identified.

26. Method according to claim 11, wherein a step (g) follows the multiplication step (e):
(g) isolation and sequencing of the DNA segment of individual virus clones which encodes the peptide or protein selected and identified as the interaction partner.

27. Method according to claim 26, wherein a step (h) follows the sequencing step (g):
(h) recombinant expression and isolation of the peptide or protein selected and identified as the interaction partner.

28. Method according to claim 27, wherein a step (i) follows the expression step (h):
(i) characterization of the bonding of the recombinantly expressed peptide or protein to the ligands used for selection in an assay.

29. Method according to claim 1, wherein immobilization of the ligands can occur directly or indirectly on the solid phase carrier.

30. Method according to claim 29, wherein a direct immobilization occurs by means of a covalent bonding of the ligands to the solid phase carrier.

31. Method according to claim 29, wherein an indirect immobilization of the ligands on the solid phase carrier is mediated by an organic intermediate layer.

32. Method according to claim 31, wherein the organic intermediate layer forms a self-assembling monolayer (SAM).

33. Method according to claim 31, wherein the organic intermediate layer is polymer-free.

34. Method according to claim 32, wherein the monolayer comprises anchor molecules.

35. Method according to claim 32, wherein the monolayer additionally comprises diluent molecules.

36. Method according to claim 1, wherein the peptide or protein molecules are selected from antibodies, enzymes, receptors, ion channels, membrane proteins and fragments thereof, which are each optionally derivatised.

37. Method according to claim 1, wherein the interaction partners presented by the virus system are encoded by DNA fragments inserted in the virus genome which form a DNA library.

38. Method according to claim 37, wherein the number of DNA fragments contained in the DNA library is at least X, with X being selected from 10², 10³, 10⁴, 10⁵, 10⁶ and 10⁷.

39. Method according to claim 37, wherein the inserted DNA fragments are isolated from cDNA or genomic DNA (gDNA), or are synthetic oligo- or polynucleotides.

40. Method according to claim 39, wherein the cDNA or gDNA stems from an eucaryotic organism.

41. Method according to claim 40, wherein the eucaryotic organism is a human being.

42. Method according to claim 40, wherein the cDNA is isolated from a differentiated tissue or a differentiated cell population.

43. Method according to claim 1, wherein the virus system is formed from viruses which use procaryotes as hosts.

44. Method according to claim 1, wherein the virus system is formed from viruses which comprise wild-type viruses or genetically modified viruses.

45. Method according to claim 44, wherein the viruses are selected from viruses having single-stranded DNA (ssDNA viruses) and double-stranded DNA (dsDNA viruses).

46. Method according to claim 45, wherein the viruses are selected from a group of bacteriophages.

47. Method according to claim 46, wherein the bacteriophage is an Escherichia coli-specific bacteriophage.

48. Method according to claim 46, wherein the bacteriophage is a filamentous bacteriophage.

49. Method according to claim 48, wherein the filamentous bacteriophage is selected from M13-, fl- and fd-phages.

50. Method according to claim 46, wherein the bacteriophage is a lytic bacteriophage.

51. Method according to claim 50, wherein the lytic bacteriophage has a polyhedral-shaped capsid.

52. Method according to claim 50, wherein the lytic bacteriophage is selected from a λ-phage, a T3-phage, a T4-phage and a TB7-phage.

53. Method according to claim 1, comprising identifying lead structures for active substance research.

54. Method according to claim 1, comprising epitope mapping.

55. Method according to claim 1, comprising proteome mapping.

56. Method according to claim 1, wherein the solid phase carrier is an SPR sensor surface support, comprising:
a plurality of SPR sensor surfaces arranged in parallel on a plane on a substrate, with ability to pass radiation for exciting surface plasmons through the substrate so that it is reflected by the SPR sensor surfaces;
separating means for separating individual SPR sensor surfaces from respectively adjacent SPR sensor areas, said separating means forming a respective cavity for each SPR sensor surface; and
a plurality of measuring areas, each measuring area comprising one or more SPR sensor surfaces, with at least one measuring area being surrounded by an isolating area, which does not have any separating means and may accommodate a sealing member to form, together with a volume element placed on the measuring area, a space above the measuring area which is isolated from adjacent measuring areas.

57. A measuring apparatus for the method of claim 1, comprising:
an SPR sensor surface support having a plurality of SPR sensor surfaces arranged in parallel on a plane on a substrate, with ability to pass radiation for exciting surface plasmons through the substrate so that it is reflected by the SPR sensor surfaces;
separating means for separating the individual SPR sensor surfaces from the respectively adjacent SPR sensor areas, said separating means forming a respective cavity for each SPR sensor surface; and
a plurality of measuring areas, each measuring area comprising one or more SPR sensor surfaces, with at least one isolated measuring area being surrounded by an isolating area with no separating means, which may accommodate a sealing member to form, together with a volume element placed on the measuring area, a space above the measuring area isolated from adjacent measuring areas.

58. The measuring apparatus of claim 57, comprising sealing members placed on isolating areas, and volume elements placed on measuring areas.
